(19) United States
(12) Patent Application Publication
(10) Pub. No.: US 2012/0309974 A1
(43) Pub. Date: Dec. 6, 2012

(54) Bianthracene compounds substituted by aromatic ring and their uses for luminescence materials

(75) Inventors: Lifei Cai, Beijing (CN); Hongyu Zhao, Beijing (CN); Lei Dai, Beijing (CN)

(73) Assignee: BEIJING AGLAIA TECHNOLOGY DEVELOPMENT CO., LTD, Beijing (CN)

(21) Appl. No.: 13/577,777

(22) PCT Filed: Feb. 9, 2011
(86) PCT No.: PCT/CN2011/070886

§ 371 Date: Aug. 8, 2012

(30) Foreign Application Priority Data
Feb. 9, 2010 (CN) 201010110501.7

(51) Int. Cl. C07C 15/28; C07D 403/10; C07D 409/10; C07C 43/275; C07C 211/54; C07C 43/20; C07C 15/38; C07C 15/60; C07D 401/14; C07F 7/08

(52) U.S. Cl. 546/256; 548/440; 549/43; 556/406; 564/427; 568/633; 585/26; 585/27

(57) ABSTRACT

The present invention relates to Aromatic ring substituted dianthracene compounds and pertains to the field of synthesis of organic light-emitting materials. Aromatic ring substituted dianthracene compounds in the formula (I) present high glass transition temperature and solution efficiency, which can be used as effective blue-light emitting host materials.

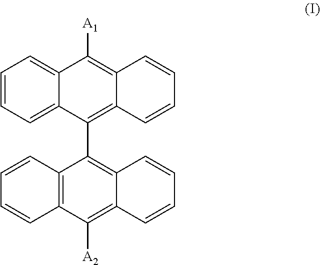

### THE TECHNICAL FIELD BELONGS

The present invention relates to Aromatic ring substituted Dianthracene compounds and pertains to the field of synthesis of organic light-emitting materials.

### TECHNICAL BACKGROUND

In 1987, Dr Ching W. Tang, et al from Kodak Company successfully prepared sandwich-type bilayer organic light-emitting diodes by using Alq₃ as light-emitting layer and Aromatic Di-Amine as hole transport layer. (Tang C. W., et al. Applied Physics Letters, 1987, 51, 913). In 1990, Burroughes J. H., et al from Cambridge University in England built light-emitting devices (LEDs) based on organic polymer (Burroughes J. H., et al. Nartue, 1990, 347, 5395). These breakthroughs greatly promoted the development of organic light-emitting technical field. Since then, scientists in different countries devoted much energy to the research and development in this field. More and more organic light-emitting materials were developed and applied. Out of all kinds of organic light-emitting materials, 9,9-Dianthranide crystal the band gap of which is about 3 eV and only can be emitted at wavelength of below 410 nm, stability in the air. And hole mobility at room temperature of Dianthranide crystal can reach 3 cm²/V·s. Consequently, Dianthranide field effect transistors get great academic interests these days. Meanwhile, its derivatives are a promising blue-emitting material, which being of importance to developing organic blue, white-light-emitting devices. (M. H. Ho, Y. S. Wu, S. W. Wen, et al., Appl. Phys. Lett., 2006, 89, 252903/1-3.).

So far, the reported Dianthranide derivatives are mainly blue-light emitting host materials with the energy level 3.1 ev, which is not conducive to the hole-transportation. (J.-H. Jou, Ch.-P. Wang, et al., Organic Electronics, 2007, 8, 29-36.) In order to raise luminous efficiency, J.-H. Jou, et al adopt total-host material, which solved problems of both holes and electron-transporting voltage barrier. The absorption spectroscopy of the two Anthracyclines of Dianthracene compounds is similar to that of separate Anthracene. (Hans Dieter Becker, Vratislav Langer, Joachim Sieler, and Hans Christian Becker, J. Org. Chem., 1992, 57 (6), 1883-1887.)

Consequently, as same as Anthracene compounds, Dianthracene can be modified structurally, raise luminous efficiency, increase of service life, and improve the stability of the devices. (Japanese patent JP200777094). Different material structures were introduced, which increased service life and improved luminescence. Moreover, Japanese patent JP2002121547 disclosed a luminescent material 9,10-Spiro Fluorene Anthracene. And the disadvantages of the luminescent material are as follows: high sublimation temperature and easy degradation. All these disadvantages above put a premium on the use of the luminescent material. With high molecular weight Dianthracene material can linked different groups at position 10, which reduced intermolecular packing and decreased sublimation temperature.

### THE CONTENTS OF THIS INVENTION

Dianthracene compounds linked with two Anthracene chromophore groups both of which are nonplanar, which causes the energy level generally higher than 3 ev. The present invention modified structurally the two positions 10, in order to improve the device parameters.

On the basis of a great amount predecessors' work as well as a lot of research, Dianthracene was modified structurally, and compounds of general formula are as below:

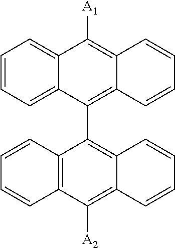

And A1, A2 is either formula (II) or (III) respectively.

(1)

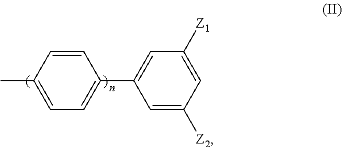

See formula (II), Z1 and Z2 are either the same or not, n=0 or 1; Z1, Z2 is respectively either substituted alkyl or un-substituted alkyl both of which linked one to fifty carbon atoms, or either substituted alkoxy or un-substituted alkoxy both of which linked one to fifty carbon atoms, or either substituted cycloalkyl or un-substituted cycloalkyl both of which linked five to fifty carbon atoms, or either substituted aralkyl or un-substituted aralkyl both of which linked six to sixty carbon atoms, or either substituted aryl or un-substituted aryl both of which linked six to sixty carbon atoms, or either substituted aryloxy or un-substituted aryloxy both of which linked six to sixty carbon atoms, or either substituted heteroaromatic group or un-substituted heteroaromatic group both of which linked five to fifty carbon atoms, or organic amine.

(2)

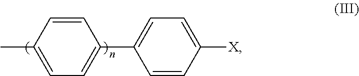

n=0 or 1, See formula (III), X is either substituted phenyl or un-substituted phenyl, or either substituted biphenyl or un-substituted biphenyl, or either substituted naphthyl or un-substituted naphthyl, or either substituted fluorenyl or un-substituted fluorenyl, or cyclic fluorenyl, anthracene spiro group, phenanthryl, anthracene, benzene fluorene moieties, perylene moieties, pyrenyl.

A1, A2 mentioned above is respectively one category of either formula (II) or formula (III), and N═O.

A1 and A2 mentioned above are the same.

Z1, Z2 mentioned above is respectively phenil, alkyl benzene, alkyl phenoxy, either substituted biphenyl or un-substituted biphenyl, or either substituted b Diphenylamine or un-substituted Diphenylamine.

Z1 and Z2 mentioned above are the same, both of which are phenyl or 4-tert-butyl-1-phenyls, note, n=0.

Z1, Z2 is respectively

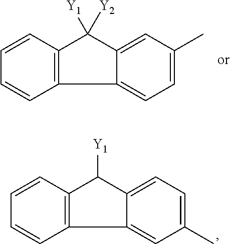

and Y1, Y2 in the formulas is C1-4 alkyl, phenyl, 4-substituted phenyl, or 1-naphthyl, note, n=0.

X is

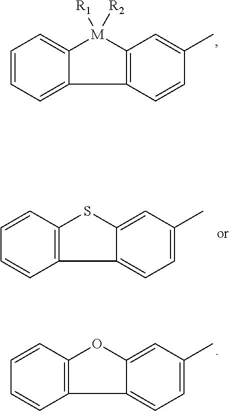

M in the formulas is carbon or silicon. R1, R2 is respectively either substituted alkyl or un-substituted alkyl both of which linked one to ten carbon atoms, either substituted phenyl or un-substituted phenyl, either substituted naphthyl or un-substituted naphthyl, either substituted biphenyl or un-substituted biphenyl, or linked with each other to be ring structure.

R1, R2 is respectively C1-4 alkyl, phenyl, or 4-methyl-1-phenyl, and n=0.

X is 4-tert-butyl-1-phenyl, 2-naphthyl, or 1-naphthyl, and n=1.

X is 2-phenanthryl, 2-benzene fluorene moieties, or 5-benzene fluorene moieties, and n=0.

In the synthesis methods of all the compounds mentioned above, Dianthracene bromide or iodide were introduced coupling reacting with either boric acid compounds A1, A2, boric acid ester or grignard reagent under alkaline and catalytic conditions.

And the introduced catalysts are as follows: Pd(PPh₃)₄, Pd(PPh₃)₂Cl₂, Pd(Ac)₂, Pd₃(dba)₂, Ni(dppf)₂, Ni(dppe)2, Ni(dppp)2 or Pd(dppf)₂. Coupling reaction solvents introduced are one of or the compounds of two maybe all the following five solvents: ether, methylbenzene, THF, 1,4-Dioxane, Dimethoxy-ethane. Alkaline solutions used for the alkaline condition are one or maybe more categories of followings: sodium carbonate, sodium bicarbonate, sodium phosphate, monometallic sodium orthophosphate, potassium carbonate, barium hydroxide, and sodium hydroxide.

All the compounds mentioned can be introduced in organic electroluminescent devices.

With the general structures, A1 and A2 can be classified into two groups that formula (II) and formula (III) with different molecular structures. A1, A2 can be any from the two groups above as long as suitable for synthesis and structural stability, then with on conditionality. Compounds of general formula can be synthesized by conventional chemical methods. And experiments and the results show that the compounds present high glass transition temperature and solution efficiency. The compounds of general formula can be used as effective blue-light emitting host materials.

“A1, A2 mentioned above is respectively one category of either formula (II) or formula (III)”, i.e. A1 and A2 are any one pairs from formula (II) and formula (III), the same kind of molecular structure, or not.

“A1, A2 is respectively one category of either formula (II) or formula (III), note, the values of n are the same.”, i.e. The molecular structure of A1 is similar to that of A2. And when both are the molecular structures of formula (II), then Z1 and Z2 are different, or when both formula (III) then X different. Namely, the compound frame structures of formula (I) are basically the same.

“A1 and A2 mentioned above are the same.” i.e. A1 and A2 are substituent groups with the same molecular structures, namely, the compounds of formula (I) are symmetrical.

The structural formulas below gave specific examples for general formulas of di-Anthracene derivative, but more than that, any compound consistent with the rules of the general formulas are of the group.

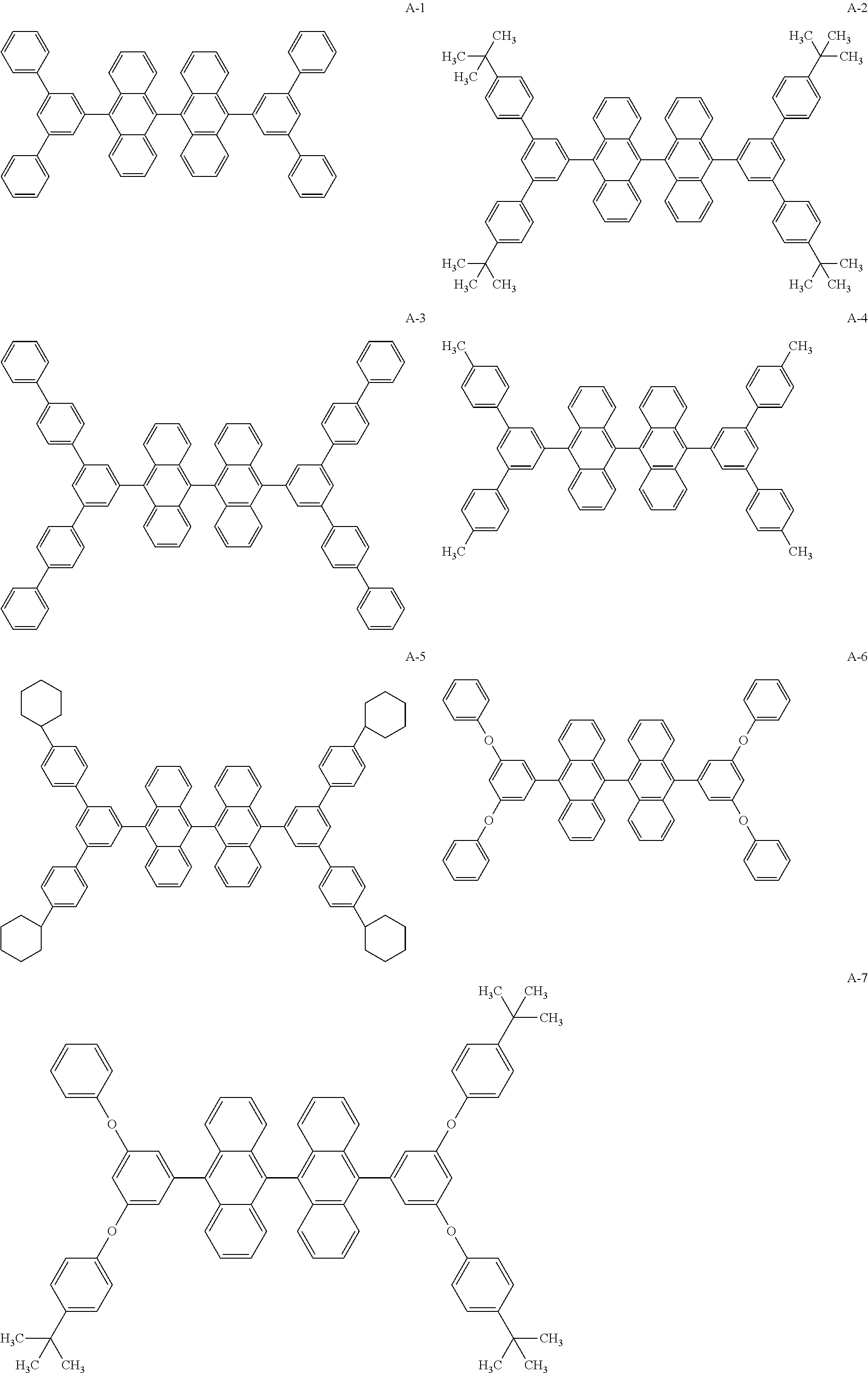

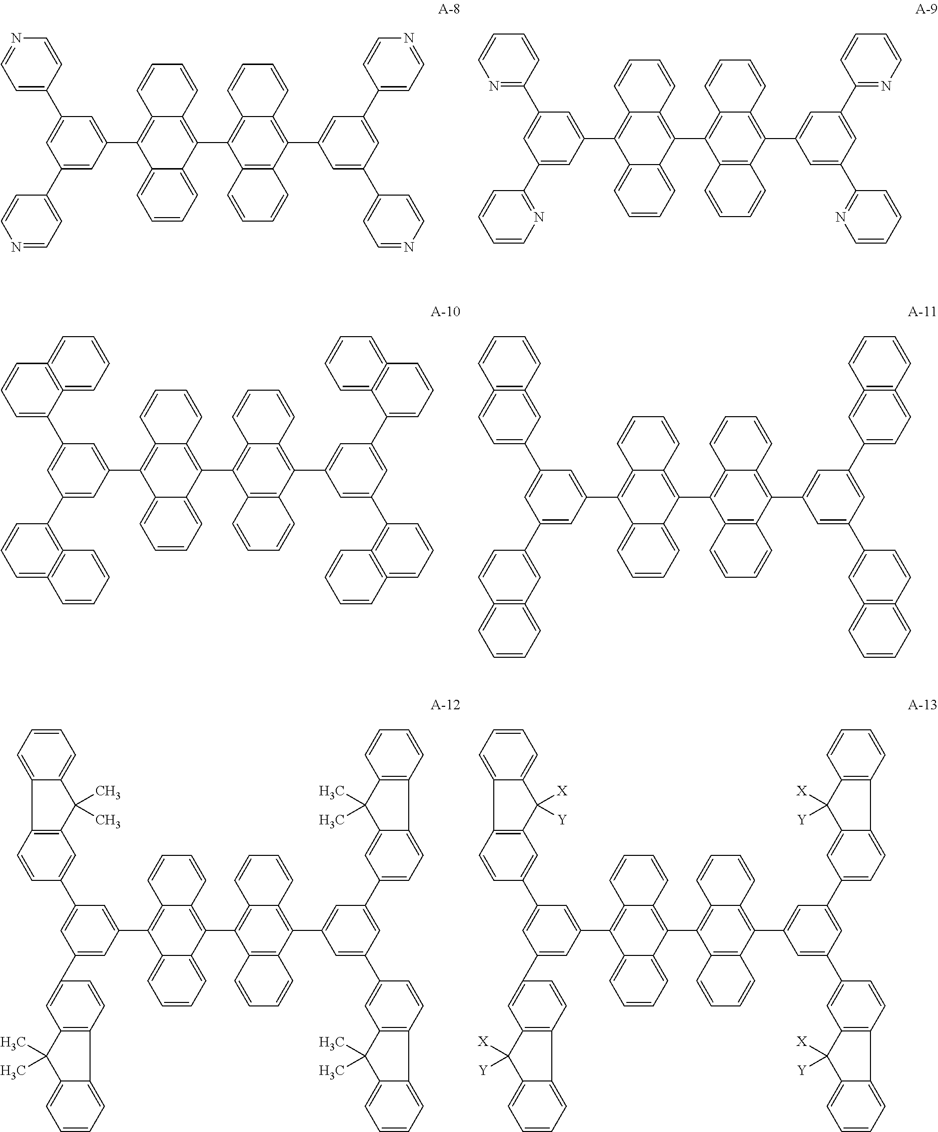

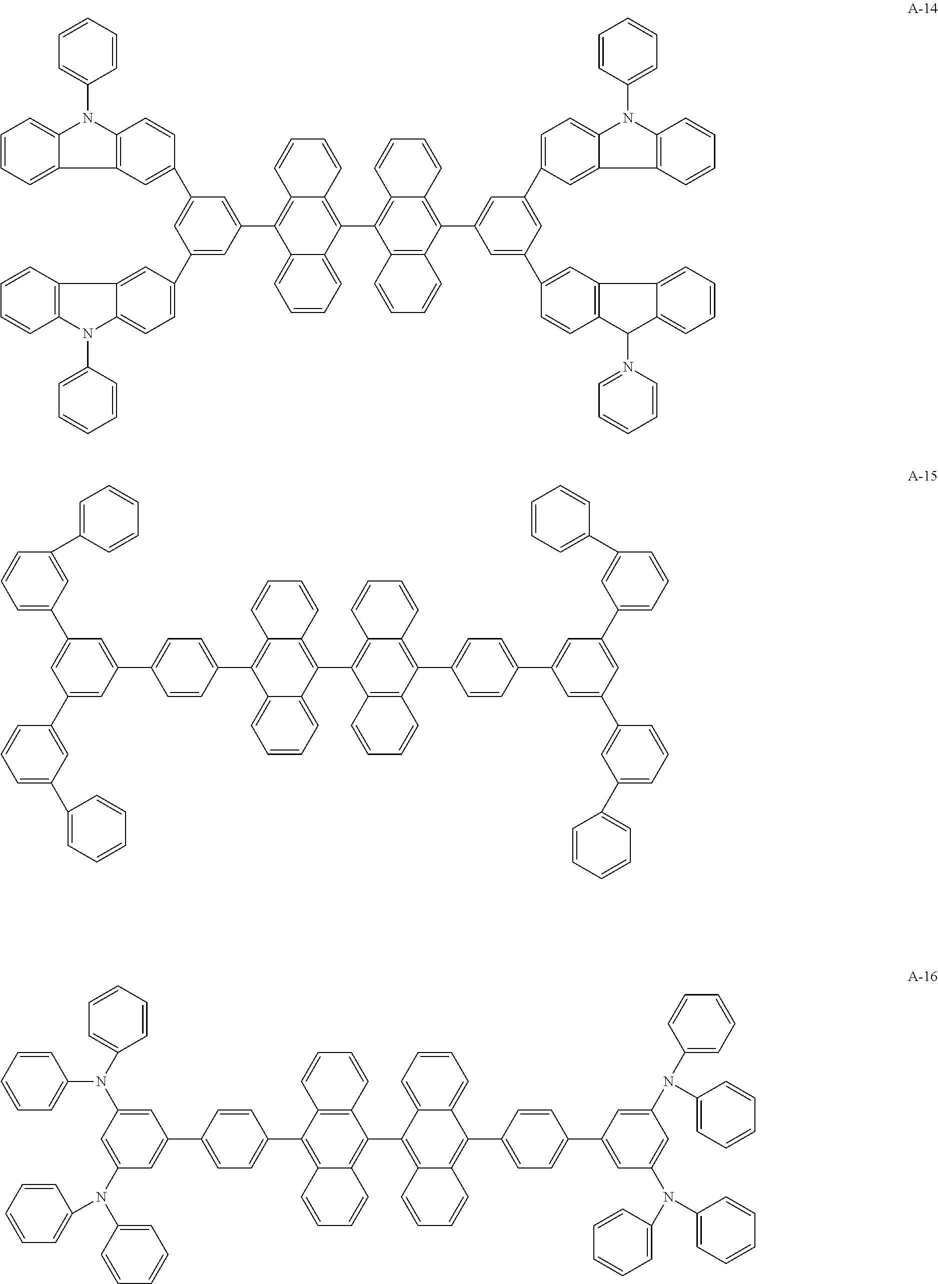

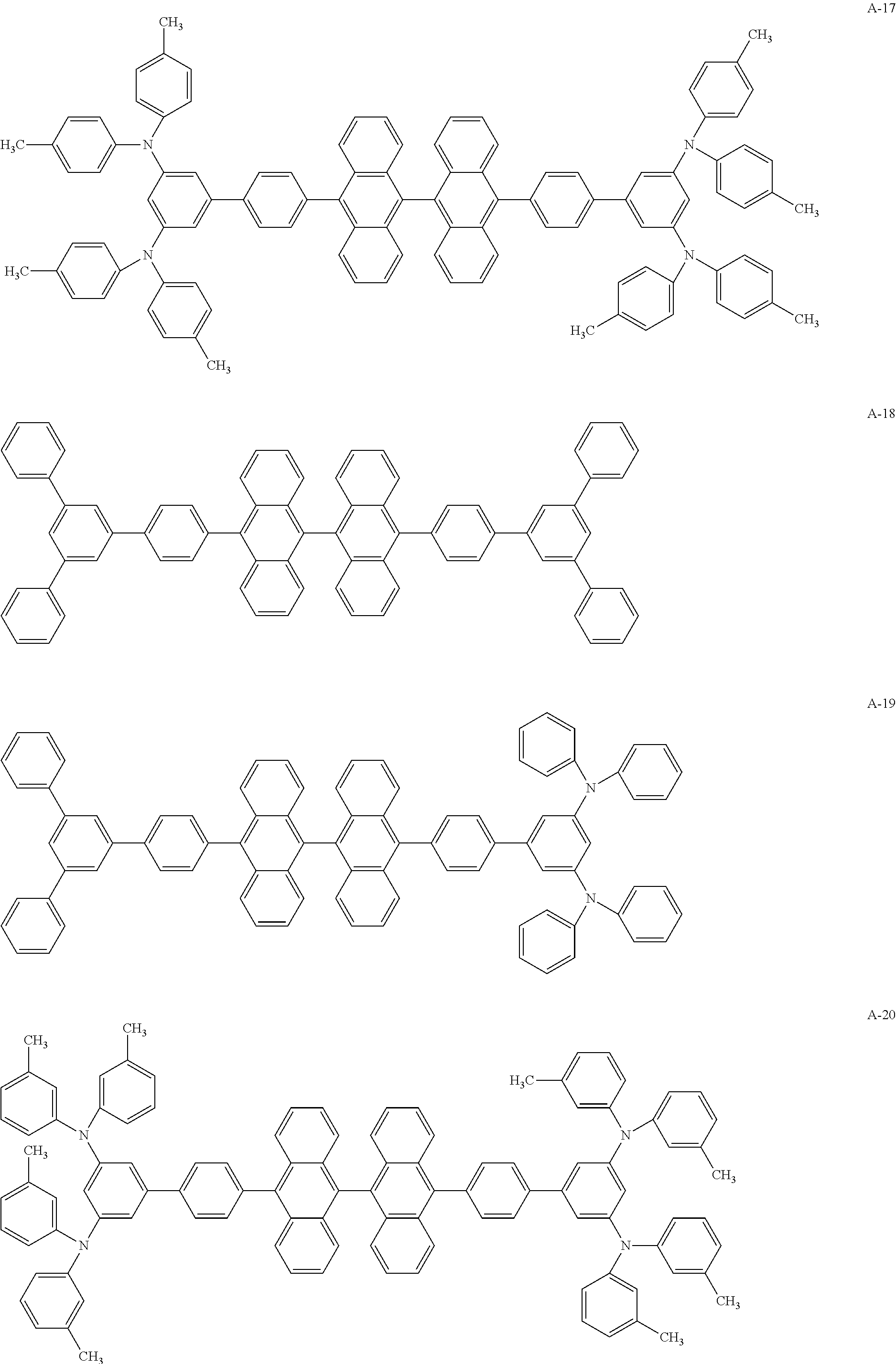

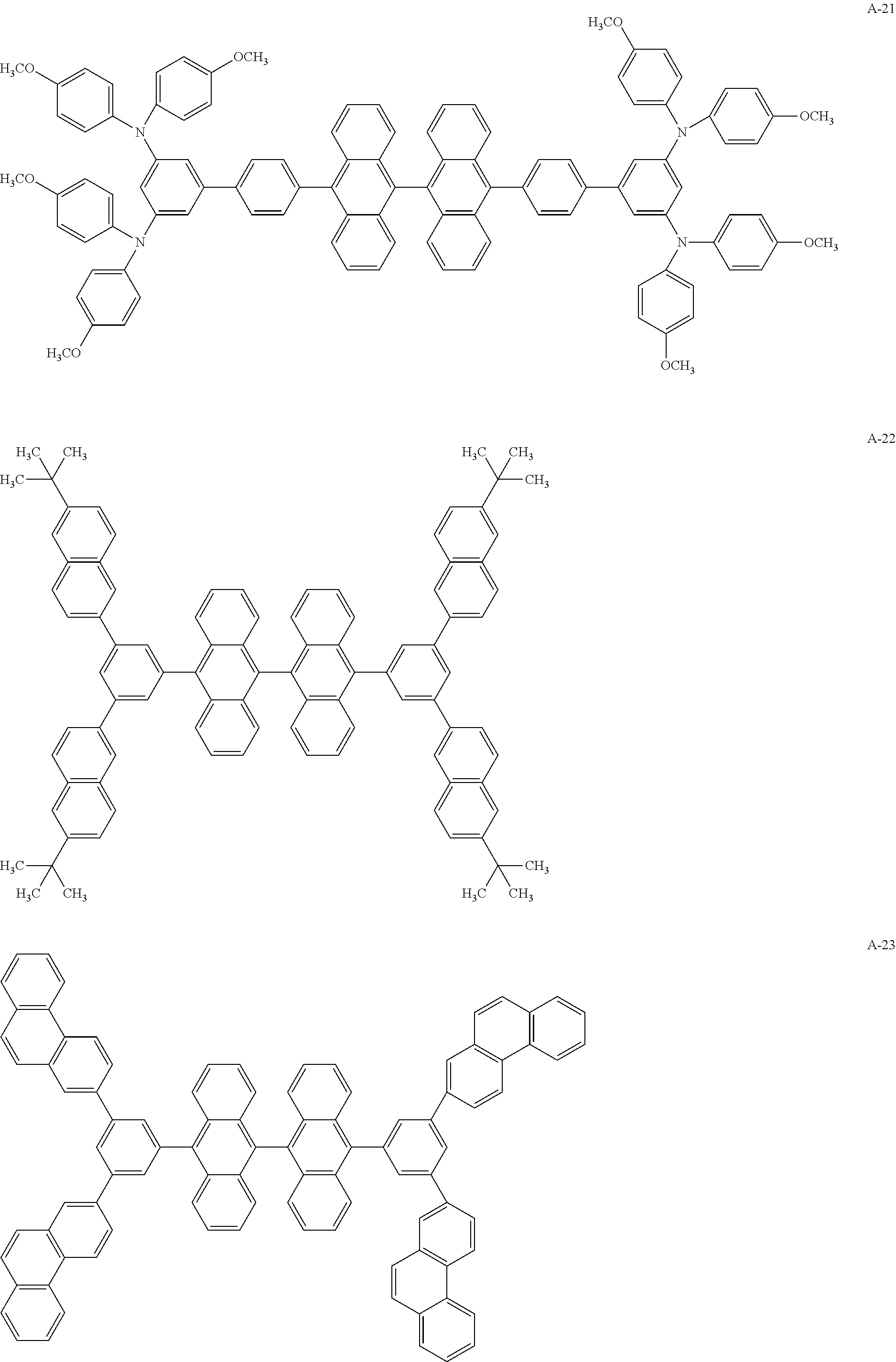

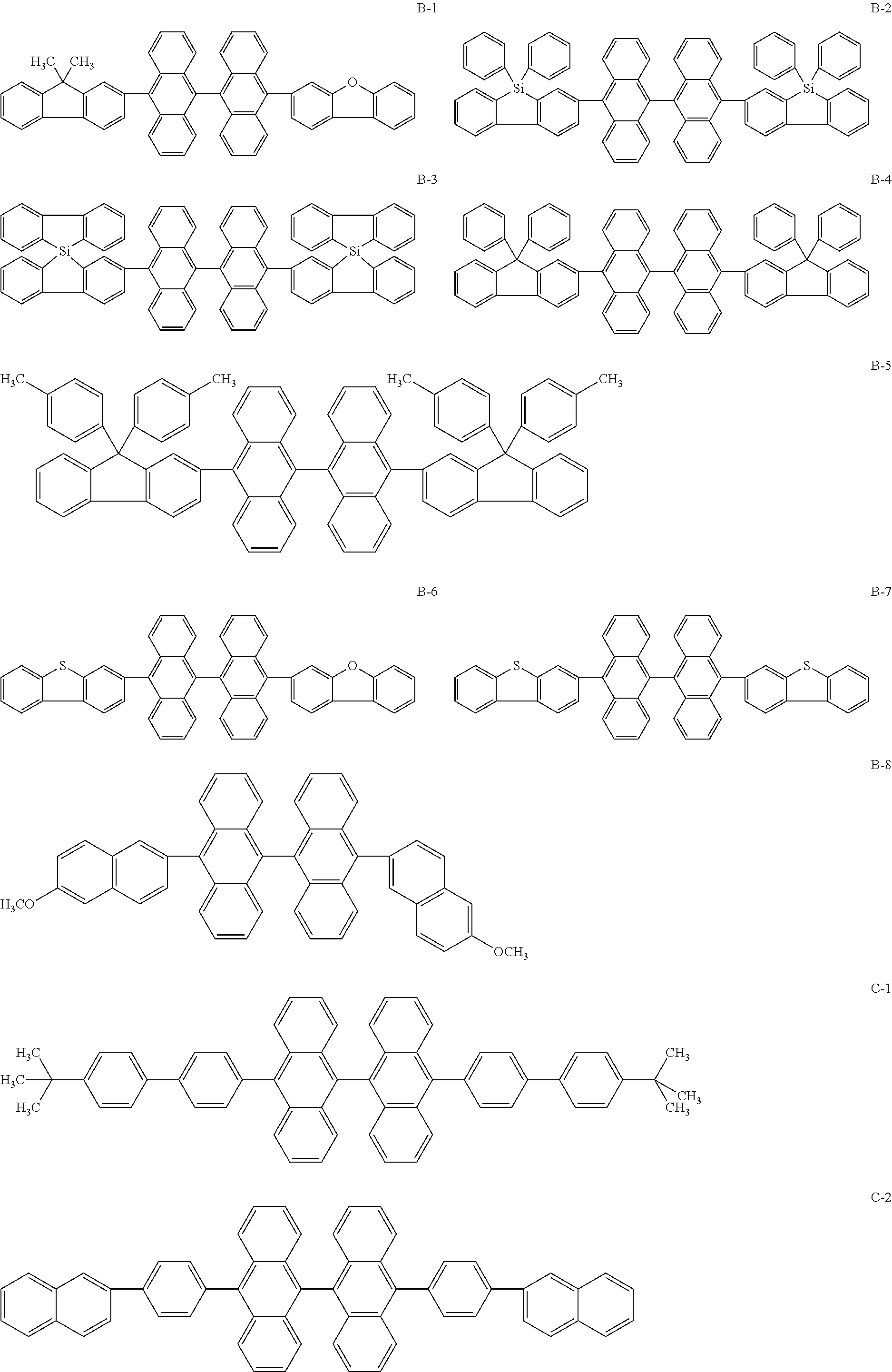

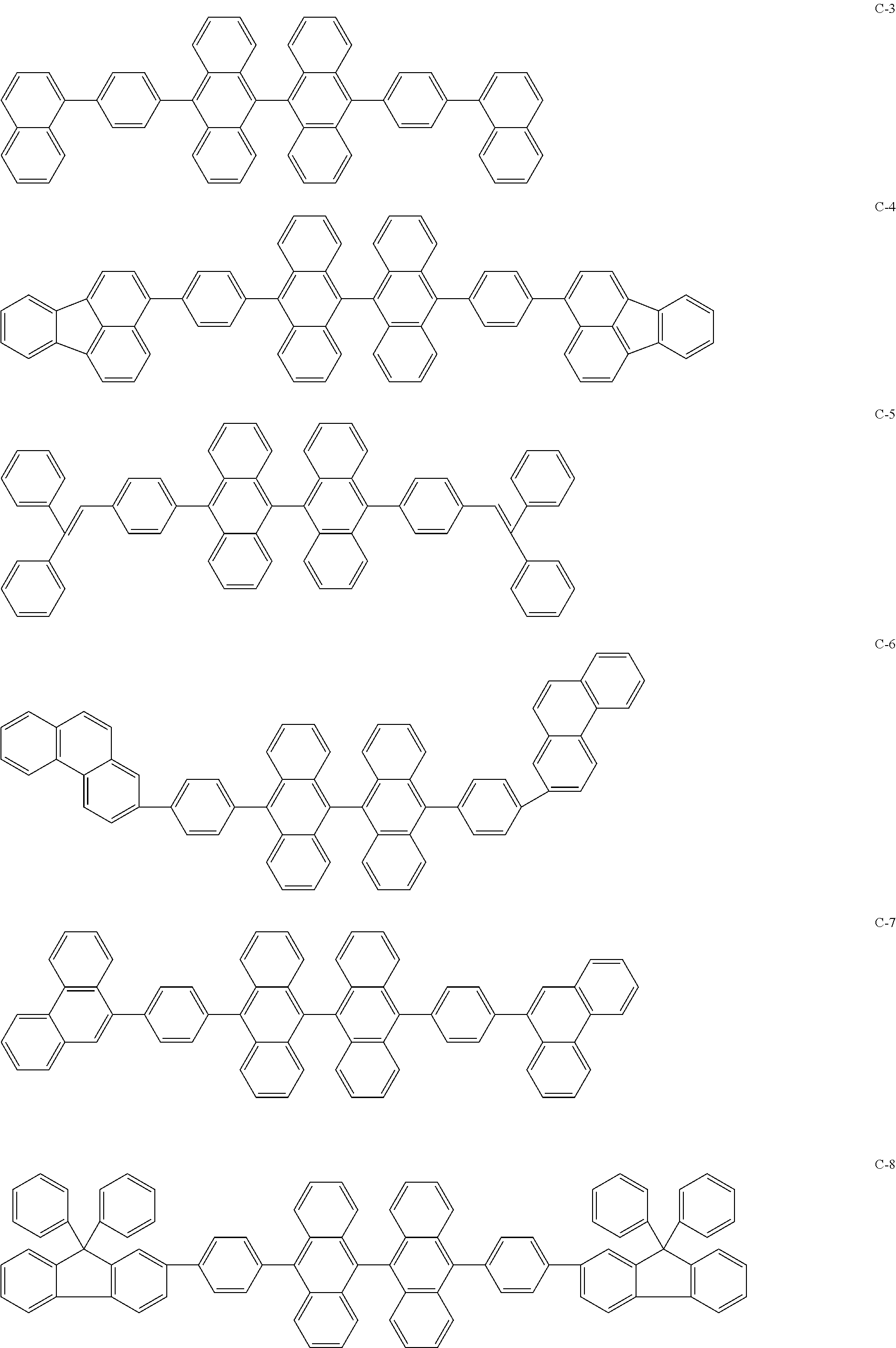

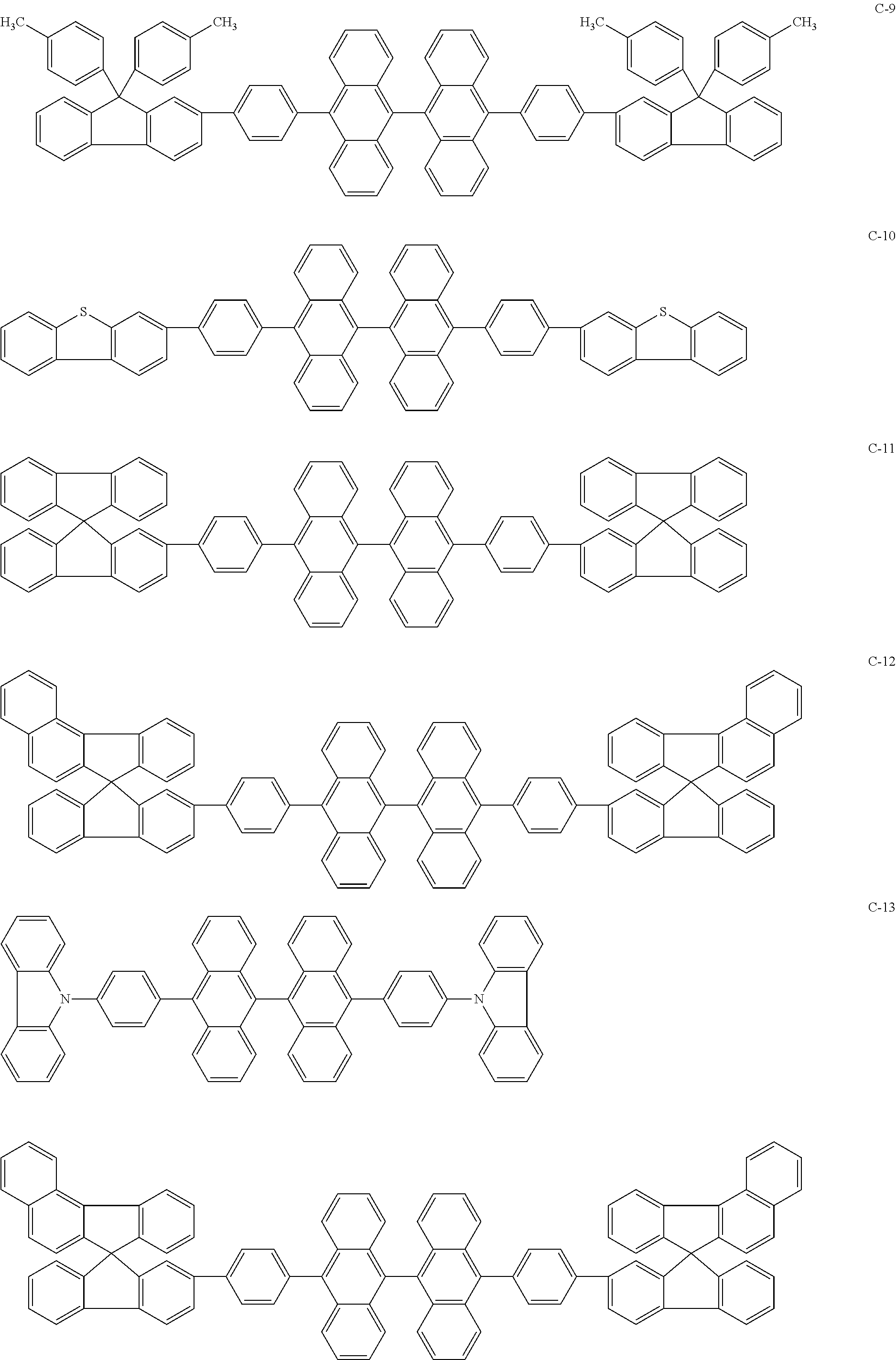

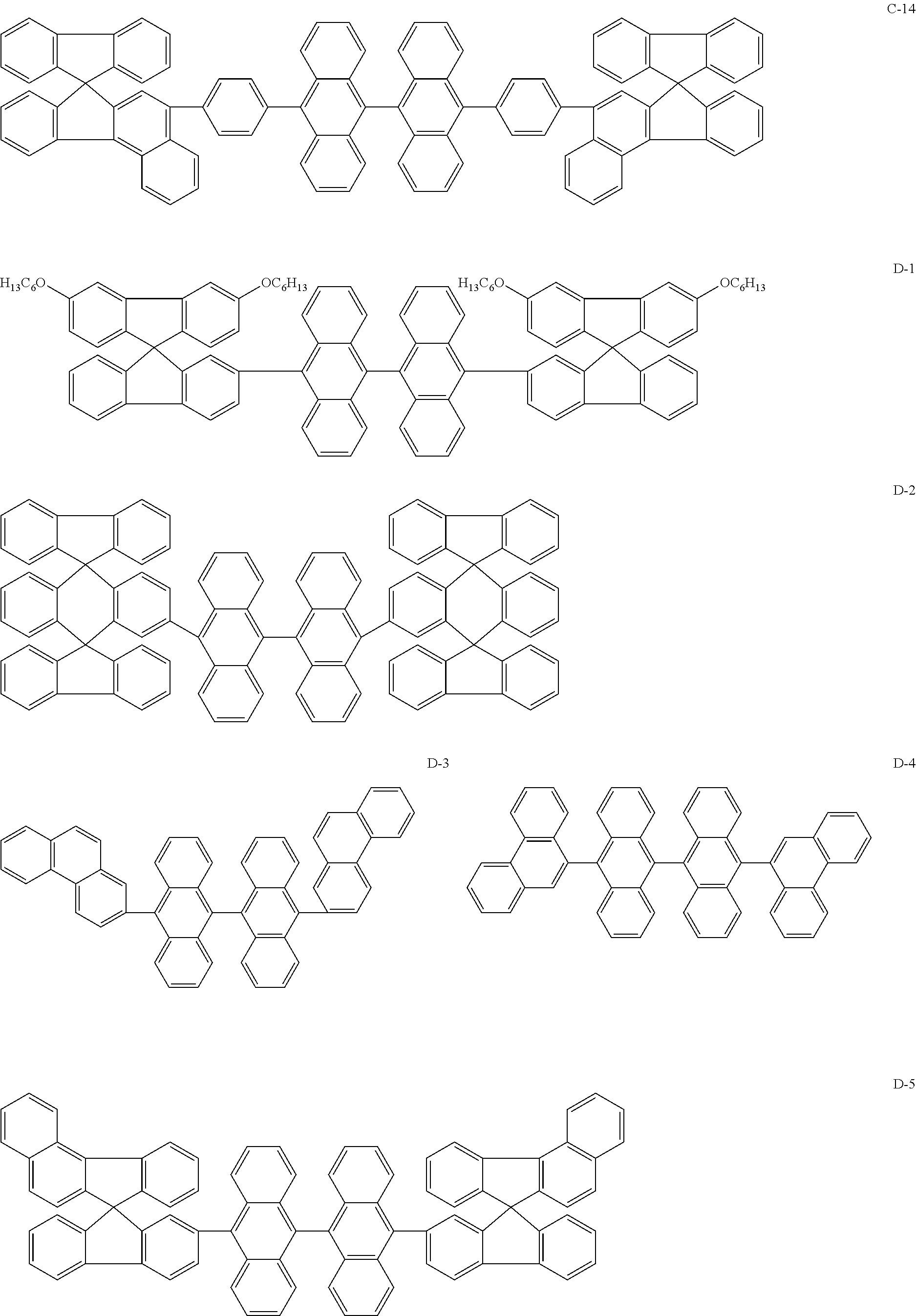

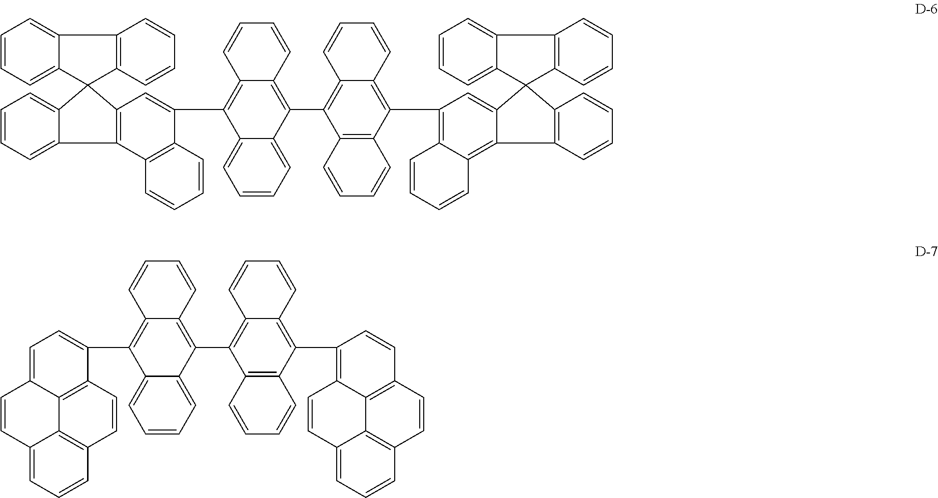

(1) X═Y=Phenyl group, (2) X═Y=4-Methylphenyl, (3) X=1-Naphthyl, Y=4-Methylphenyl (4) 2,2′-biphenyl

### DESCRIPTION OF FIGURES

FIG. 1 shows the photoluminescence (PL) and ultra-violet spectra (UV) of B-4.

FIG. 2 shows the photoluminescence (PL) and the electroluminescence (EL) of B-4.

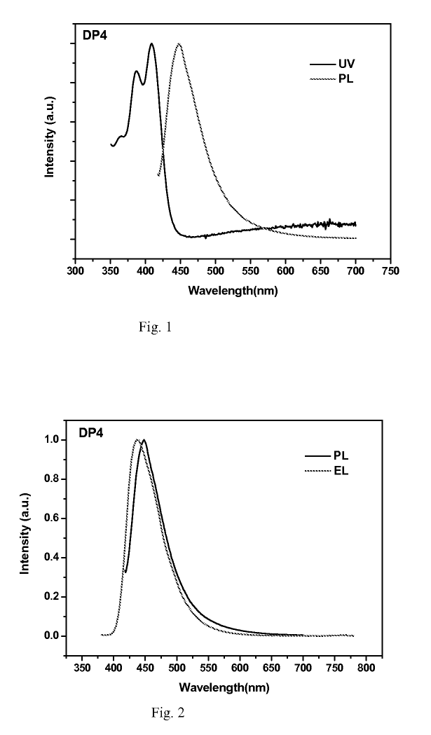

### THE DETAILED METHOD AND BASIC PROCESSES

Some details will have to be further elaborated as we go along with patent embodiments.

### Embodiment 1

### The Synthesis of A-1

### The synthesis of 3,5-DiphenylBroMobenzene

Added 2.6 g (0.11 mol) of freshly prepared Magnesium chips to 500 ml of three-neck flask. Under the Argon gas field, dripped in 62 ml of THF solution dissolved with 12 mL (0.114 mol) of Bromobenzene, and controlled the dropping rate to moderate the violence of the reaction. After, refluxed for about 0.5 hour, and stopped when Magnesium chips disappeared. Then the gray pasty-liquid reached. Dripped in 75 ml of THF solution containing 8.8 g (0.2 mol) of 2,4,6-tribromolodobenzene, and then under the Argon gas field stirred 3 hours at room temperature, followed 1.5 hours of refluxed. After cooling, poured the reaction solution into the freezing diluted HCl, turned acidic and stable overnight. Then after removed THF from screen-out organic layers by using evaporation method, residues were extracted by Methylene Chloride, then rinsed in turn, by Na₂SO₃ and water, and dried by MgSO₄. After removed Methylene Chloride, 6 g of gray white solid substance left with 80% yield rate. Recrystallized by Ethanol, 4.1 g of white crystalline solid reached, melting point: 108.8˜109.4° C., MS (m/z): 308, yield rate: 66%.

### The Synthesis of the Target Product A-1

Added 6 g (20 mmol around) of (3,5-Diphenylphenyl) boronic acid (see (U.S. Pat. No. 6,361,886)), 3.3 g (10 mmol around) of 10,10′-Dibromo Bianthracene and 35 mg of Tetrakis (Triphenylphosphine) Palladium [Pd (PPh₃)₄] to a four-neck flask. Under the Argon gas field, added 10 ml 2M of NaHCO₃, 40 ml of Methylbenzene, and 15 ml Ethanol, then heated, refluxed and stirred for more than 4 hours then stopped. Then after air cooled to the room temperature, in turn followed, extracted by Ether, combined organic phases, rinsed, dried by anhydrous Magnesium Sulfate, and removed the solvent by the evaporation way. Then reached coarse products were further purified with 100 to 200-mesh silica gel column chromatography, using Petroleum Ether and Dichloromethane as eluent with 5:1 volume ratio. And 7 g of yellow solid substances reached, 73% of yield rate.

δ: 8.04 (S, 2H), 7.86˜7.92 (m, 4H), 7.76 (d, J=8.00 Hz, 12H), 7.48 (t, J=7.20 Hz, 8H), 7.34˜7.42 (m, 8H).

### Embodiment 2

### The Synthesis of A-2

### The synthesis of 3,5-bis(4-Tert-Butylphenyl)

Added 100 ml of THF, 0.95 g (3 mmol around) of 1,3,5-Tribromobenzene and 100 mg of Tetrakis (Triphenylphosphine) Palladium [Pd (PPh₃)₄] to a four-neck flask. Under the Argon gas field, dripped in around 6.2 mmol of 4-tert-Butylphenylmagnesium, then heated and refluxed. After stirred for more than 2 hours stopped the reaction and air cooled to the room temperature. Then quenched with 10% of diluted HCl, followed by liquid separated, extracted, combined organic phases, rinsed, dried by anhydrous Magnesium Sulfate, and removed the solvent by the evaporation way. Then reached coarse products were further purified with 100 to 200-mesh silica gel column chromatography, using Petroleum Ether as eluent. And 0.6 g of white solid substances reached, 42% of yield rate.

HNMR (400 MHz, CDCl3) δ (ppm): 7.71 (1H, s), 7.68 (2H, s), 7.55 (4H, d, J=8.40), 7.49 (4H, d, J=8.36), 1.38 (18H, s), EI MS m/z=422.

### The synthesis of 3,5-bis(4-Tert-Butylphenyl) Phenylboronic acid

Added 45 g (107 mmol around) of 3,5-bis(4-Tert-Butylphenyl) and 10 ml of anhydrous THF to a four-neck flask. Under the Argon gas field, cooled to around −80° C., slowly dripped in 80 ml 1.6M of Lithiumn-Butyl for 0.5 hour, and then stirred another half an hour at −80° C. Dissolved 24 g of Trimethylborate with 300 ml of anhydrous THF, then dripped in 28 ml around 230 mmol of this solution slowly for about 0.5 hour at −80° C. Then air warmed up the reaction system, stirred and stable overnight. Added in 150 ml 2M of HCl, and stirred for 1 hour. Then extracted by Methylene Chloride, combined organic phases, followed rinsed, and dried by anhydrous Magnesium Sulfate. And after removed the solvent by the evaporation way, the pale yellow coarse products left. Then reached coarse products were further purified with 100 to 200-mesh silica gel column chromatography, using Petroleum Ether as eluent first, then rinsed by Ethyl Acetate, reached 0.23 g (60 mmol around) of white solid substances, 55% of yield rate around.

### The Synthesis of the Target Product A-2

Added 7.8 g (20 mmol around) of 3,5-bis(4-Tert-Butylphenyl), 3.3 g (10 mmol around) of 10,10′-Dibromo Bianthracene and 35 mg of Tetrakis (Triphenylphosphine) Palladium [Pd (PPh₃)₄] to a four-neck flask. Under the Argon gas field, added 10 ml 2M of Na₂CO₃, followed with 40 ml of Methylbenzene and 15 ml of Ethanol. Heated, refluxed, and stirred for more than 24 hours, then stop the reaction and air cooled to the room temperature. Then extracted by Ether, combined organic phases, followed by rinsed, dried by anhydrous Magnesium Sulfate. And after removed the solvent by the evaporation way, the coarse products left. Then reached coarse products were further purified with 100 to 200-mesh silica gel column chromatography, using Petroleum Ether and Dichloromethane as eluent with 5:1 volume ratio. And 8 g of yellow solid substances reached, 75% of yield rate around.

H NMR (400 MHz, CDCl₃) δ (ppm): 8.04 (2H, t, J=1.68), 7.87-7.90 (4H, m), 7.69-7.74 (12H, m), 7.50 (8H, d, J=6.64), 7.34-7.38 (4H, m), 1.38 (36H, s), MALDI-TOF MS m/z=1035.

### Embodiment 2

### The Synthesis of A-14

### The synthesis of N-Phenyl Carbazole-3-Boric Acid

Added 322.2 g of N-phenyl-3-Bromocarbazole (commercial product) in 2 L of THF, filled with Nitrogen gas, stirred, and cooled to −78° C. Then dripped 400 ml of Lithiumn-Butyl by controlled the dropping rate for 2 hours. 290 g of Triisopropyl Borate was diluted with 300 ml of THF, then dripped in reaction solution at −78° C. The reaction lasted for 2 hours with temperature constant. Then gradually warmed up to the room temperature, and reacted for overnight. After cooled to −10° C., dripped in 500 ml 10% of diluted HCl, stirred fort hours, followed with liquid separated and concentrated, 210 g of Boric acid reached, 70% of yield rate.

### The synthesis of 3,5-bis(N-phenyl Carbazole) Bromobenzene

Added 1.8 g (6.2 mmol around) of N-Phenyl Carbazole-3-Boric Acid, 0.95 g (3 mmol around) of 1,3,5-Tribromobenzene and 100 mg Tetrakis (Triphenylphosphine) Palladium [Pd (PPh₃)₄] to a four-neck flask. Under the Argon gas field, added in 3 ml of 2M of Na₂CO₃, followed with 10 ml of Methylbenzene and 3 ml of Ethanol. Heated, refluxed, and stirred for more than 8 hours, then stop the reaction and air cooled to the room temperature. Then extracted by Dichloromethane, combined organic phases, followed by rinsed, dried by anhydrous Magnesium Sulfate. And after removed the solvent by the evaporation way, the coarse products left. Then reached coarse products were further purified with 100 to 200-mesh silica gel column chromatography, using Petroleum Ether as eluent. And 1.6 g of white solid substances reached, 60% of yield rate around.

### The synthesis of 3,5-bis(N-Phenyl Carbazole) Phenylboronic acid

According to the synthetic method for 3,5-bis(4-Tert-Butylphenyl), 3,5-bis(N-Phenyl Carbazole) Phenylboronic acid can be reached with 60% of yield rate. MALDI-TOF MS m/z=677.52.

### The synthesis of A-14

Added 13.55 g (20 mmol around) of 3,5-bis(N-Phenyl Carbazole) Phenylboronic acid, 3.3 g (10 mmol around) of 10,10′-Dibromo Bianthracene and 70 mg of Tetrakis (Triphenylphosphine) Palladium [Pd (PPh₃)₄] to a four-neck flask. Under the Argon gas field, added 10 ml 2M of Na₂CO₃, followed with 40 ml of Methylbenzene and 15 ml of Ethanol. Heated, refluxed, and stirred for more than 24 hours, then stop the reaction and air cooled to the room temperature. Then extracted by Ether, combined organic phases, followed by rinsed, dried by anhydrous Magnesium Sulfate. And after removed the solvent by the evaporation way, the coarse products left. Then reached coarse products were further purified with 100 to 200-mesh silica gel column chromatography, using Petroleum Ether and Dichloromethane as eluent with 5:1 volume ratio. And 10.3 g of yellow solid substances reached, 70% of yield rate around. MALDI-TOF MS m/z=1471.78.

### The Synthesis of B-4

### 9,9-Diphenyl-2-Fluorene Boric acid

Dissolved 4.6 g of 9,9-Diphenyl-2-Bromo Fluorene (see J. AM. CHEM. SOC. 2002, 124, 11576-11577) in 100 ml of THF, stirred under Nitrogen gas field and cooled to −78° C. Constantly dripped it to the volume of 7 ml, and after stirred for 2 hours, dripped in 4 ml of Tributyl Borate. The reaction lasted for 4 hours with temperature constant, then warmed up to the room temperature and stirred overnight. After cooled to −10° C., dripped in 10% diluted HCl, the PH value reached 1, and stirred for 2 hours. Then later, followed with liquid separated, extracted by Methylene Chloride, concentrated, and further purified by silica gel column chromatography, 3 g of product can be reached, with 80% of purity.

### The Synthesis of B-4

Added 7 g (20 mmol around) of 9,9-Diphenyl-2-Fluorene Boric acid, 3.3 g (10 mmol around) of 10,10′-Dibromo Bianthracene, 70 mg of [Pd₂(dba)₂] and 0.5 g of tri-tert-butylphosphine to a four-neck flask. Under the Argon gas field, added 10 ml 2M of Na₂CO₃, followed with 40 ml of Methylbenzene and 15 ml of Ethanol. Heated, refluxed, and stirred for more than 24 hours, then stop the reaction and air cooled to the room temperature. Then extracted by Ether, combined organic phases, followed by rinsed, dried by anhydrous Magnesium Sulfate. And after removed the solvent by the evaporation way, the coarse products left. Then reached coarse products were further purified with 100 to 200-mesh silica gel column chromatography, using Petroleum Ether and Dichloromethane as eluent with 5:1 volume ratio. And 6.9 g of yellow solid substances reached, 70% of yield rate around. MALDI-TOF MS m/z=987

### The Synthesis of C-1

According to the synthetic method for A-14, 3,5-bis(N-Phenyl Carbazole) Phenylboronic acid was replaced by 4-Tert-Butyl Diphenyl Boric acid (commercial product) to synthesize B-4, pale yellow solid substances can be reached with 80% of yield rate. MALDI-TOF MS m/z=771.

### The Synthesis of C-2

Added 4.96 g (20 mmol around) of 4-(2-Naphthyl) Phenylboronic acid (see J. Org. Chem. 2000, 65, 6319-6337), 3.3 g (10 mmol around) of 10,10′-Dibromo Bianthracene, 70 mg of [Pd₂(dba)₂] and 0.5 g of tri-tert-butylphosphine to a four-neck flask. Under the Argon gas field, added 10 ml 2M of Na₂CO₃, followed with 40 ml of Methylbenzene and 15 ml of Ethanol. Heated, refluxed, and stirred for more than 6 hours, then stop the reaction and air cooled to the room temperature. Then extracted by Ether, combined organic phases, followed by rinsed, dried by anhydrous Magnesium Sulfate. And after removed the solvent by the evaporation way, the coarse products left. Then reached coarse products were further purified with 100 to 200-mesh silica gel column chromatography, using Petroleum Ether and Dichloromethane as eluent with 5:1 volume ratio. And 5.3 g of yellow solid substances reached, 70% of yield rate around. MALDI-TOF MS m/z=759

### The Synthesis of D-3

Added 4.44 g (20 mmol around) of 2-Philippine Boric acid (see Thin Solid Films 516 (2008) 8717-8720), 3.3 g (10 mmol around) of 10,10′-Dibromo Bianthracene, 70 mg of [Pd (Ac)₂] and 0.5 g of tri-tert-butylphosphine to a four-neck flask. Under the Argon gas field, added 10 ml 2M of Na₂CO₃, followed with 40 ml of Methylbenzene and 15 ml of Ethanol. Heated, refluxed, and stirred for more than 6 hours, then stop the reaction and air cooled to the room temperature. Then extracted by Ether, combined organic phases, followed by rinsed, dried by anhydrous Magnesium Sulfate. And after removed the solvent by the evaporation way, the coarse products left. Then reached coarse products were further purified with 100 to 200-mesh silica gel column chromatography, using Petroleum Ether and Dichloromethane as eluent with 5:1 volume ratio. And 5.3 g of yellow solid substances reached, 70% of yield rate around. MALDI-TOF MS m/z=706.869

### The Synthesis of D5

Added 96 g (20 mmol around) of Spiro[Fluorene-based-7,9′-Phenylfluorone]-2-Boric acid (see J. AM. CHEM. SOC. 2002, 124, 11576-11577), 3.3 g (10 mmol around) of 10, 10′-Dibromo Bianthracene, 70 mg of [Pd (Ac)₂] and 0.5 g of tri-tert-butylphosphine to a four-neck flask. Under the Argon gas field, added 10 ml 2M of Na₂CO₃, followed with 40 ml of Methylbenzene and 15 ml of Ethanol. Heated, refluxed, and stirred for more than 6 hours, then stop the reaction and air cooled to the room temperature. Then extracted by Ether, combined organic phases, followed by rinsed, dried by anhydrous Magnesium Sulfate. And after removed the solvent by the evaporation way, the coarse products left. Then reached coarse products were further purified with 100 to 200-mesh silica gel column chromatography, using Petroleum Ether and Dichloromethane as eluent with 5:1 volume ratio. And 5.3 g of yellow solid substances reached, 70% of yield rate around. MALDI-TOF MS m/z=1083.3

¹H NMR (500 MHz, CDCl₃) d 8.75-8.73 (d, 1H), 8.35-8.33 (d, 1H), 8.13-8.10 (d, 1H), 8.00-7.95 (m, 3H), 7.90-7.85 (d, 1H), 7.77 (s, 1H), 7.67-7.65 (m, 1H), 7.60-7.54 (m, 9H), 7.53-7.40 (m, 4H), 7.24-7.15 (m, 7H), 7.02-7.01 (d, 2H), 6.86 (s, 1H), 6.79-6.78 (d, 1H).

### The Synthesis of D-6

Added 7.9 g (20 mmol around) of Spiro[Fluorene-7,9′-Phenylfluorone]-S-Boric acid (see J. AM. CHEM. SOC. 2002, 124, 11576-11577), 3.3 g (10 mmol around) of 10, 10′-Dibromo Bianthracene, 70 mg of [Pd (Ac)₂] and 0.5 g of tri-tert-butylphosphine to a four-neck flask. Under the Argon gas field, added 10 ml 2M of Na₂CO₃, followed with 40 ml of Methylbenzene and 15 ml of Ethanol. Heated, refluxed, and stirred for more than 6 hours, then stop the reaction and air cooled to the room temperature. Then extracted by Ether, combined organic phases, followed by rinsed, dried by anhydrous Magnesium Sulfate. And after removed the solvent by the evaporation way, the coarse products left. Then reached coarse products were further purified with 100 to 200-mesh silica gel column chromatography, using Petroleum Ether and Dichloromethane as eluent with 5:1 volume ratio. And 5.3 g of yellow solid substances reached, 70% of yield rate around. MALDI-TOF MS m/z=1083.3

¹H NMR (500 MHz, CDCl₃) 9.03-9.01 (d, J 8.43 Hz, 1H), 8.60-8.58 (d, J 8.19 Hz, 1H,), 8.19-8.17 (d, J 8.44 Hz, 1H), 7.97-7.95 (d, J 8.40 Hz, 1H), 7.90-7.88 (t, 4H,), 7.86-7.83 (t, 4H,), 7.81-7.78 (t, 1H,), 7.62-7.60 (d, 1H,), 7.59-7.57 (d, 2H,), 7.54-7.51 (d, 4H,), 7.50-7.47 (d, 4H,), 7.47-7.45 (d, 4H,), d 7.43-7.40 (d, 2H,), 7.40-7.39 (d, 2H,), 7.16-7.14 (d, 2H,), 7.14-7.13 (d, 1H,), 6.89-6.88 (d, 2H,), 6.88 (d, 1H,).

1. Aromatic ring substituted dianthracene compounds see formula (I), 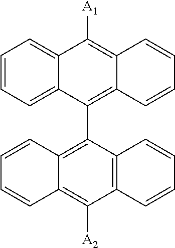
And A1, A2 is either formula (II) or (III) respectively.
(1) 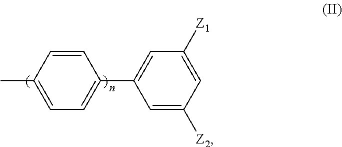
See formula (II), Z1 and Z2 are either the same or not, n=0 or 1; Z1, Z2 is respectively either substituted alkyl or un-substituted alkyl both of which linked one to fifty carbon atoms, or either substituted alkoxy or un-substituted alkoxy both of which linked one to fifty carbon atoms, or either substituted cycloalkyl or un-substituted cycloalkyl both of which linked five to fifty carbon atoms, or either substituted aralkyl or un-substituted aralkyl both of which linked six to sixty carbon atoms, or either substituted aryl or un-substituted aryl both of which linked six to sixty carbon atoms, or either substituted aryloxy or un-substituted aryloxy both of which linked six to sixty carbon atoms, or either substituted heteroaromatic group or un-substituted heteroaromatic group both of which linked five to fifty carbon atoms, or organic amine.
(2) 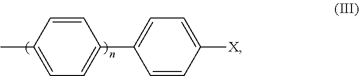
n=0 or 1, See formula (III), X is either substituted phenyl or un-substituted phenyl, or either substituted biphenyl or un-substituted biphenyl, or either substituted naphthyl or un-substituted naphthyl, or either substituted fluorenyl or un-substituted fluorenyl, or cyclic fluorenyl, anthracene spiro group, phenanthryl, anthracene, benzene fluorene moieties, perylene moieties, pyrenyl.

2. According to the claim 1, in the aromatic ring substituted dianthracene compounds formula mentioned above, A1, A2 is respectively one category of either formula (II) or formula (III), note, the values of n are the same.

3. According to the claim 2, in the aromatic ring substituted dianthracene compounds formula mentioned above, A1 and A2 are the same.

4. According to the claim 3, in the aromatic ring substituted dianthracene compounds formula mentioned above, Z1, Z2 is respectively phenil, alkyl benzene, alkyl phenoxy, either substituted biphenyl or
un-substituted biphenyl, or either substituted b diphenylamine or un-substituted diphenylamine.

5. According to the claim 4, in the aromatic ring substituted dianthracene compounds formula mentioned above, Z1 and Z2 are the same, both of which are either phenils or 4-tert-butyl-1-phenyls, note, n=0.

6. According to the claim 3, in the aromatic ring substituted dianthracene compounds formula mentioned above, Z1, Z2 is respectively 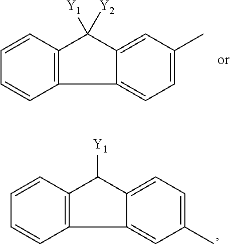
and Y1, Y2 in the formulas is
C1-4 alkyl, phenyl, 4-substituted phenyl, or 1-naphthyl, note, n=0.

7. According to the claim 3, in the aromatic ring substituted dianthracene compounds formula mentioned above, X is 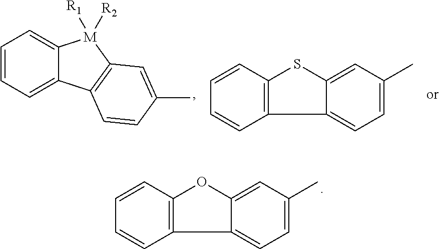
M in the formulas is carbon or silicon. R1, R2 is respectively either substituted alkyl or un-substituted alkyl both of which linked one to ten carbon atoms, either substituted phenyl or un-substituted phenyl, either substituted naphthyl or un-substituted naphthyl, either substituted biphenyl or un-substituted biphenyl, or linked with each other to be ring structure.

8. According to the claim 7, in the aromatic ring substituted dianthracene compounds formula mentioned above, R1, R2 is respectively C1-4 alkyl, phenyl, or 4-methyl-1-phenyl, and n=0.

9. According to the claim 7, in the aromatic ring substituted dianthracene compounds formula mentioned above, X is 4-tert-butyl-1-phenyl, 2-naphthyl, or 1-naphthyl, and n=1.

10. According to the claim 3, in the aromatic ring substituted dianthracene compounds formula mentioned above, X is 2-phenanthryl, 2-benzene fluorene moieties, or 5-benzene fluorene moieties, and n=0.

11. According to claim 1, in the synthesis methods of all the compounds mentioned above, dianthracene bromide or iodide were introduced coupling reacting with either boric acid compounds A1, A2, boric acid ester or grignard reagent under alkaline and catalytic conditions.

12. According to the synthesis method of the claim 11, the introduced catalysts are as follows: Pd(PPh₃)₄, Pd(PPh₃)₂Cl₂, Pd(Ac)₂, Pd₃(dba)₂, Ni(dppf)₂, Ni(dppe)2, Ni(dppp)2 or Pd(dppf)2. Coupling reaction solvents introduced are one of or the compounds of two maybe all the following five solvents: ether, methylbenzene, THF, 1,4-dioxane, dimethoxy-ethane. Alkaline solutions used for the alkaline condition are one or maybe more categories of followings: sodium carbonate, sodium bicarbonate, sodium phosphate, monometallic sodium orthophosphate, potassium carbonate, barium hydroxide, and sodium hydroxide.

13. According to claim 1, all the compounds mentioned can be introduced in organic electroluminescent devices.
